(19) United States
(12) Patent Application Publication
(10) Pub. No.: US 2024/0237901 A1
(43) Pub. Date: Jul. 18, 2024

(54) INTEGRATED TEMPERATURE SENSOR

(71) Applicant: KONINKLIJKE PHILIPS N.V., Eindhoven (NL)

(72) Inventor: Steven Peter Groen, Atkinson, NH (US)

(21) Appl. No.: 18/289,009

(22) PCT Filed: Apr. 29, 2022
(86) PCT No.: PCT/EP2022/061600

§ 371 Date: Oct. 31, 2023

Related U.S. Application Data
(60) Provisional application No. 63/184,887, filed on May 6, 2021.

(51) Int. Cl. A61B 5/01; A61B 5/00; A61B 5/0205; A61B 5/08; A61B 5/1455

(52) U.S. Cl. CPC A61B 5/01; A61B 5/02055; A61B 5/0816; A61B 5/14552; A61B 5/6819; A61B 5/0008; A61B 2560/0252

(57) ABSTRACT

An integrated sensor (110) includes a temperature sensor (202) configured to measure the absolute temperature of internal air exhaled from the body at the nasal ala, and the absolute temperature of external air inhaled into the body at the nasal ala. A system (100) is adapted to estimate an internal (core) temperature and to estimate ambient temperature at an application site of the integrated sensor (110).

### BACKGROUND

Vital signs are core patient characteristics that are typically taken by healthcare professionals including nurses, nursing aides, and clinical technicians. Different vital signs may be, individually and typically intermittently, captured in different ways such as by asking questions about pain and/or by using a blood pressure cuff, a pulse oximeter, a thermometer and/or a stethoscope.

Known ways for ascertaining vital signs often require a plurality of sensor devices. Among other drawbacks, the use of multiple sensors requires vital signs to be measured in series, resulting in inefficient use of health care professionals' time. For example, the need to sequentially take vital signs requires time to set up each of the various sensor devices, to gather the vital signs, to disconnect the various sensors, and to clean the various sensors and connections thereto.

What is needed, therefore, is a system for monitoring various vital signs of a patient that overcomes at least the shortcomings of known devices noted above.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified schematic view of a system for integrated nasal ala SPO2 and temperature sensing, in accordance with a representative embodiment.

FIG. 2A is a perspective view of an integrated sensor for integrated nasal ala SPO2 and temperature sensing, in accordance with a representative embodiment.

FIG. 2B is an exploded view of the integrated sensor of FIG. 2A, in accordance with a representative embodiment.

FIG. 3 is a perspective view of an integrated sensor for integrated nasal ala SPO2 and temperature sensing and adapter, in accordance with a representative embodiment.

FIG. 4 is a graph of temperature versus time based on readings from an integrated sensor of the present teachings.

FIG. 5 is a flow-chart of a method for integrated nasal ala SPO2 and temperature sensing, in accordance with a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation, and methods of manufacture may be omitted so as to avoid obscuring the description of the representative embodiments. Nonetheless, systems, devices, materials, and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments, and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of known apparatuses and methods may be omitted so as to not obscure the description of the example embodiments. Such methods and apparatuses are within the scope of the present disclosure.

As described herein, an integrated sensor may be used to measure multiple vital signs. In accordance with a representative embodiment, the nasal ala is used as the location for placement of the integrated sensor for multiple reasons including the presence of the airway (nasal passages) and the profound presence of blood due to the arterial plexus. Notably, while various representative embodiments described below relate to an integrated sensor that may be used at or around the nasal ala, the integrated sensors of the present teachings are contemplated for use in to measure temperature of a patient's skin. Regardless of the location for placement of the integrated sensor, as will become clearer as the present description continues, vital signs may be measured directly by the integrated sensor, or may be indirectly derived from patient characteristics measured directly by the integrated sensor.

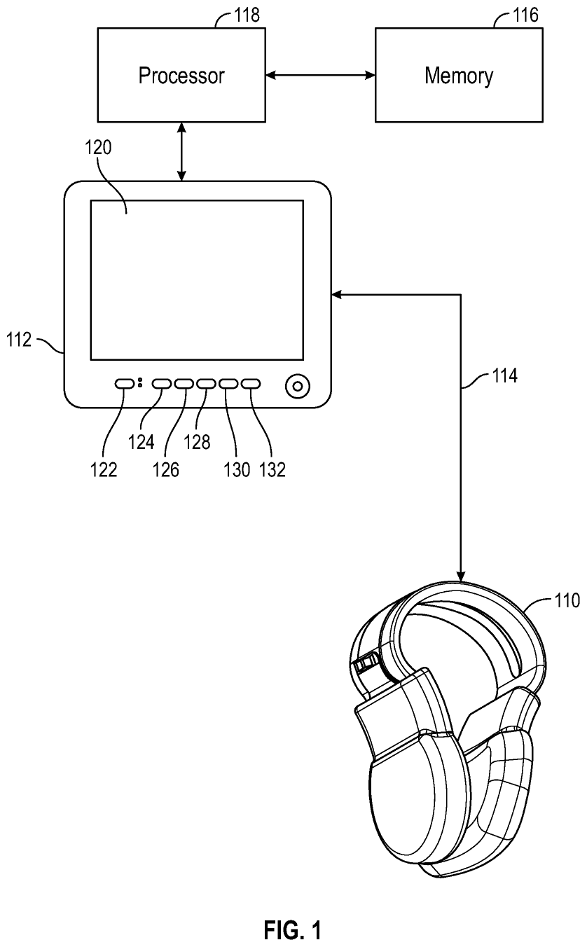

FIG. 1 illustrates a system 100 for integrated SPO2 and temperature sensing, in accordance with a representative embodiment.

The system 100 in FIG. 1 comprises an integrated sensor 110, a patient monitor 112, a connection 114, a processor 118 and a memory 116.

As described below in connection with various representative embodiments the integrated sensor 110 is adapted to measure a variety of vital signs of a patient. For example, the integrated sensor is adapted to measure oxygen saturation, the temperature of ambient air at the nasal ala, the temperature of exhaled air exiting a nasal passage at the application site, and the temperature of the skin (e.g., on the cheek).

As described more fully below, the connection 114 may be an electrical cable that connects the integrated sensor 110 to the patient monitor 112. Generally, the cable comprises a signal transmission line and a power line. Notably, the use of an electrical cable for connection 114 is merely illustrative, and other types of connections are contemplated to provide communication between the integrated sensor 110 and the patient monitor 140. By way of illustration, instead of the electrical cable, the connection 114 may be via an optical fiber transmission line. Alternatively, the connection between the integrated sensor 110 and the patient monitor 140 may be wireless. Just by way of illustration, a wireless link compliant with IEEE 802.11(x) is contemplated to effect the connection between the integrated sensor 110 and the patient monitor 140.

To measure temperature, the integrated sensor 110 must receive electrical power. As such, while optical fiber and wireless communication are contemplated for transmission of data between the patient monitor 140 and the integrated sensor 110, connection 114 must also provide power for operation of the integrated sensor. In certain embodiments, the transmission of power from the patient monitor 140 may be effected by a separate power line connected between the patient monitor 140 and the integrated sensor 110. Moreover, the present teachings contemplate a separate (portable) device comprising a battery to connect to the integrated sensor. In such a representative embodiment, the separate device may be held or attached to a patient providing power to the integrated sensor 110. Data from the integrated sensor 110 could then be transmitted wirelessly from a transmitter disposed in the integrated sensor 110 or from the separate device. Alternatively, for example when the connection 114 is wireless, the present teachings contemplate one of a number of wireless power techniques (WPTs) that are near field (within approximately one wavelength (2) of the antenna of the transmission system), or far field at a distance greater than approximately one wavelength (2) of the antenna of the transmission system.

In combination, the memory 116 and processor 118 may be referred to as a controller. In certain representative embodiments, the memory 116 and the processor 118 may be components disposed in the patient monitor 112. Alternatively, the memory 116 and the processor 118 may be separate elements from the patient monitor 112. Just by way of illustration, the connection 114 may comprise a Smart Cable (e.g., serial RS232 cable), which in-turn comprises the memory 116 and the processor 118.

The system 100 can be deployed as, or in, a device that in turn is in an integrated system that includes additional devices. In an embodiment, the system 100 can be implemented using electronic devices that provide voice, video or data communication. The system 100 may be a self-contained system, or more generally, may be deployed as a part of a computer network. In this case, the processor 118 and memory 116 may be components of a server or as a client user computer in a server-client user network environment, or as a peer computer system in a peer-to-peer (or distributed) network environment. Further, while the system 100 is illustrated in the singular, the term “system” shall also be taken to include any collection of systems or sub-systems that individually or jointly execute a set, or multiple sets, of software instructions to perform one or more computer functions.

The patient monitor 112 comprises a display 120. The display 120 may be one of a number of known types of displays, including but not limited to a light emitting diode (LED), a liquid crystal display (LCD), an organic light emitting diode (OLED), a flat panel display, a solid-state display, or a cathode ray tube (CRT), for example.

The patient monitor 112 also comprises at least a first interface 122, a second interface 124, a third interface 126, a fourth interface 128, a fifth interface 130 and sixth interface 132. The various interfaces are selected for specific functions useful in the gathering of data from the integrated sensor 110. For example, one of the interfaces may be a Fourier artifact suppression technology (FAST) socket used to connect a cable (in connection 114) to a circuit board of the patient monitor 112 such as an SPO2 circuit board. More generally, the first˜sixth interfaces 122-132 may be configured to connect a variety of other electronic devices to the patient monitor 140.

The patient monitor 112 may comprise a stationary computer, a mobile computer, personal computer (PC), a laptop computer, a tablet computer, a mobile/cellular telephone, or any other machine capable of executing a set of software instructions (sequential or otherwise) that specify actions to be taken by that machine.

The processor 118, which is tangible and non-transitory, is representative of one or more processors. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 118 (and other processors) of the present teachings is an article of manufacture and/or a machine component.

The processor 118 is configured to execute software instructions stored in the memory 116 to perform functions as described in the various embodiments herein. The processor 118 may be a general-purpose processor or may be part of an application specific integrated circuit (ASIC). The processor 118 may also be (or include) a microprocessor, a microcomputer, a processor chip, a controller, a microcontroller, a digital signal processor (DSP), a state machine, or a programmable logic device. The processor 118 may also be (or include) a logical circuit, including a programmable gate array (PGA) such as a FPGA, or another type of circuit that includes discrete gate and/or transistor logic. The processor 118 may be (or include) a central processing unit (CPU), a graphics processing unit (GPU), or both. Additionally, the processor 118 may comprise multiple processors, parallel processors, or both. Multiple processors may be included in, or coupled to, a single device or multiple devices.

The memory 116 may comprise a main memory, a static memory, or both, where the memories may communicate with each other via a bus (not shown). The memory 116 described herein are tangible storage mediums that can store data and executable instructions, and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time.

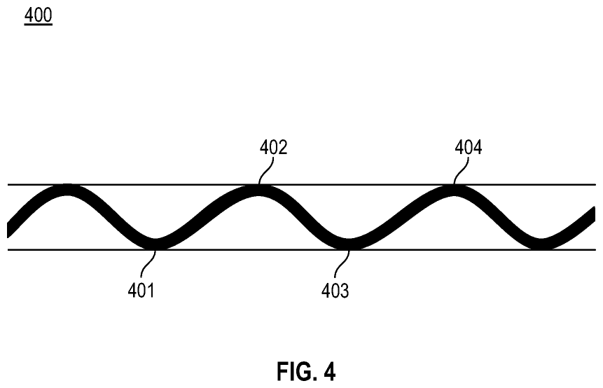

The memory 116 of the present teachings is an article of manufacture and/or machine component. The memory 116 includes one or more computer-readable mediums from which data and executable instructions (e.g., to carry out the processes described in connections with FIG. 4) can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known to one of ordinary skill in the art. Memories of the present teachings may be volatile or non-volatile, secure and/or encrypted, unsecure and/or unencrypted. The patient monitor 112, the memory 116 and the processor 118 may be housed within or linked to a workstation (not shown) such as a computer or another assembly of one or more computing devices, a display/monitor, and one or more input devices (e.g., a keyboard, joysticks and mouse) in the form of a standalone computing system, a desktop or a tablet, for example.

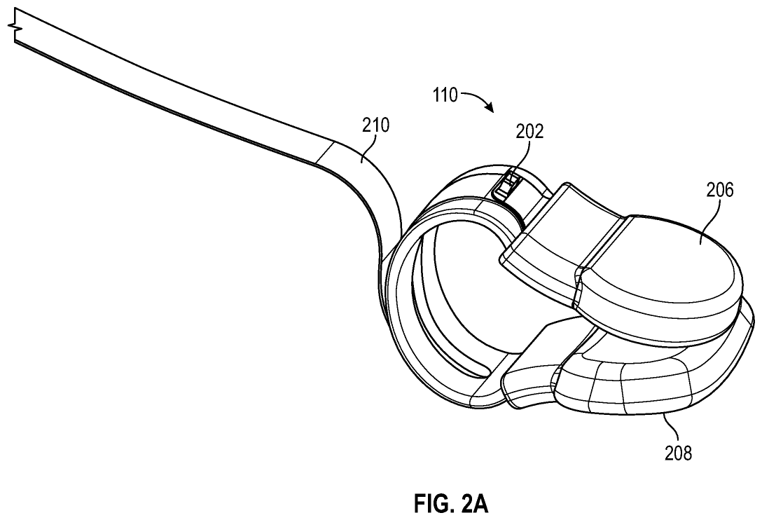

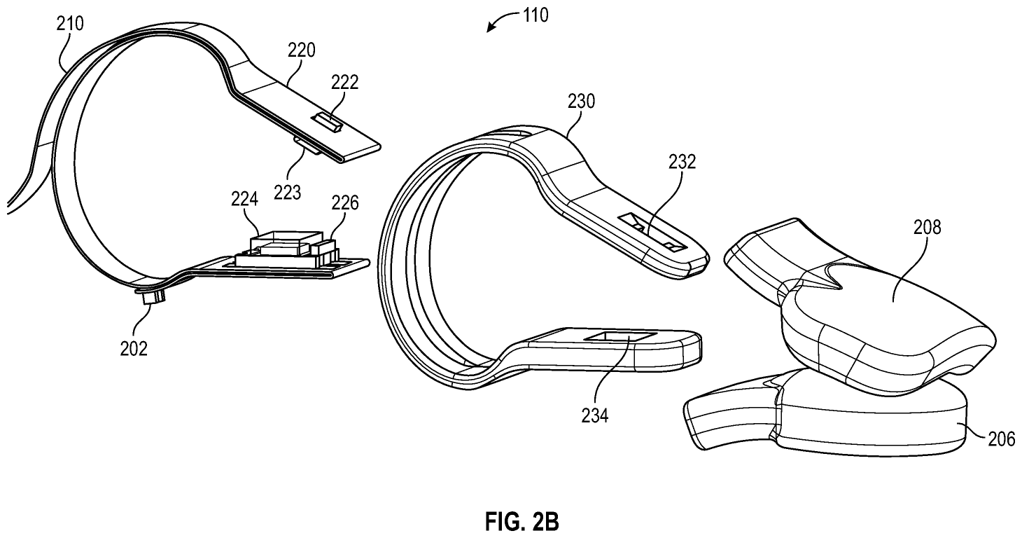

The memory 116 stores instructions that are executed by the processor 118 to implement aspects of methods described herein. For example, the processor 118 may apply instructions to the measurements received from a light receiver (not shown in FIG. 1) and temperature sensor(s) of the integrated sensor 110. The processor 118 may create time series of measurements and generate displays of the time series for the display 120. Additionally, the processor 118 may determine changes in measurements and compare changes to predetermined rules, such as when SPO2 measurements decrease below a predetermined level or decrease by a predetermined amount within a predetermined period of time. The measurements from the light receiver (see FIG. 2B) and the temperature sensor(s) of the integrated sensor 110 may also be combined with other measurements or data to produce other clinical analysis.

The instructions stored in the memory 116 and executed by the processor 118 may include one or more software programs used to display time series including time series of measurements captured by the integrated sensor 110. For example, the circuit board of the patient monitor 112 may be configured to convert analog signals of the oxygen saturation to digital signals, and the processor 118 may be configured to process the digital signals of the oxygen saturation by executing the instructions to generate the time series and other types of information. Additionally, the circuit board of the patient monitor 112 may be configured to convert analog signals of the temperatures to digital signals, and the processor 118 may be configured to process the digital signals of the temperatures by executing the instructions to generate the time series and other types of information.

Among other functions, and as described more fully below, the processor 118 may be configured to determine one of a variety of useful measurements of a patient based on measurements made by the integrated sensor 110. Specifically, the processor 118 may determine changes in ambient (room) temperature, changes in the difference between internal temperature and skin temperature, or changes in core temperature. As alluded to above, and as described more fully below, these changes in temperature measurements may be used by the processor 118 to determine a respiratory rate, readings of arterial blood gas (ABG) and/or end-tidal CO2 (EtCO2). As a result, the integrated sensor 110 may directly provide or indirectly be used to provide a number of basic vital signs on a continuous basis.

FIG. 2A is a perspective view of an integrated sensor 110 for integrated nasal ala SPO2 and temperature sensing, in accordance with a representative embodiment. As will become clearer as the present description continues, the integrated sensor 110 is contemplated for use in the system 100 described above. Notably, various aspects and details of the integrated sensor 110, and its functionality may be common to those described above in connection with representative embodiments of FIG. 1. These details may not be repeated below in the interest of clarity of description.

The integrated sensor 110 comprises a temperature sensor 202, a light detector (not shown in FIG. 2A), and a light emitting diode (LED) (not shown in FIG. 2A), which are disposed in pads 206, 208. In certain embodiments, the integrated sensor 110 further comprises a flexible circuit 210, which is adapted to connect the integrated sensor to connection 114. Various aspects of the flexible circuit 210 are described, for example, in U.S. Pat. No. 10,390,715, the disclosure of which is specifically incorporated herein by reference, and a copy of which is included in this filing.

The pads 206, 208 are adapted to be attached by friction fit to a nasal ala (not shown) of a patient. As described more fully below in connection with FIG. 2B, sensors may be disposed beneath the pads 206, 208. As described more fully below, when the pads 206, 208 are attached to the nasal ala, a variety of measurements can be made in the region surrounding the nasal ala. Notably, the location where the pads are attached to the nasal ala forms an application site of the integrated sensor 110. The application site encompasses the region at or near the point(s) of attachment to the nasal ala, and the region nearby that is within measurement range of the various sensors deployed in the integrated sensor 110. By way of example, the temperature sensor 202 may be used to measure the temperatures of the air inhaled and the air exhaled by a patient, the temperature of the ambient near the integrated sensor 110, the temperature of the skin where the integrated sensor 110 is attached to the patient, and the SPO2. As will become clearer as the present teachings continue, the various sensors and measurements made thereby are thus at the application site of the integrated sensor 110.

The temperature sensor 202 according to the present teachings is adapted to determine an absolute temperature and not merely relative temperature or merely changes in temperature. Specifically, the temperature sensor 202 generally does not comprise a thermistor, which generally only measures changes in the temperature and does not measure absolute temperature. Rather, and for purposes of illustration, the temperature sensor 202 may be one or more of a variety of absolute temperature sensor including, but not limited to a thermocouple, an application specific integrated circuit (ASIC), and a silicon/semiconductor temperature sensor. In one illustrative embodiment, the temperature sensor 202 may comprise a MAX 31820 commercially available from Maxim Integrated Products R, San Jose, CA (USA).

The temperature sensor 202 may include sensors, sensor components, monitors or other elements to measure one or several temperatures at the nasal ala. The temperature sensor 202 is illustratively configured to measure inhaled air temperature and exhaled air temperature continuously or continually. These measured temperatures may be used to derive other temperatures (e.g., estimated internal/core temperature of a patient, and estimated room temperature). Illustratively, known modeling techniques that are stored in memory 116 as instructions and executed by the processor 118 could be used to predict core temperatures based on the average or adjusted [what does adjusted mean here] average temperature from the temperature sensor 202, where the adjusted average temperature is of exhaled air that is a mix of alveolar air in the lungs and room air mixed together. Exhaled air can be correlated but it not a direct representative of core temp Furthermore, variations in the room air and exhaled air temperatures may be indicators of depth of breathing since shallow breaths create less temperature change than deep breaths. Notably, the present teachings further contemplate the inclusion of additional temperature sensors in the integrated sensor 110 to measure, for example skin temperature, and ambient temperature directly.

FIG. 2B is an exploded view of the integrated sensor of FIG. 2A, in accordance with a representative embodiment. Notably, various aspects and details of the integrated sensor 110, and its functionality may be common to those described above in connection with representative embodiments of FIGS. 1-2A. These details may not be repeated below in the interest of clarity of description.

The flexible circuit 210 is connected to a first component 220 of the integrated sensor 110. The first component 220 comprises the temperature sensor 202, an ambient temperature sensor 222, light emitting diodes (LEDs) 223, light detector 224 and a skin temperature sensor 226.

The first component 220 is adapted to be disposed over a second component 230. Moreover, pads 206, 208 are disposed over portions of the second component 230.

When the first component 220 is disposed over and connected to the second component 230, a first opening 232 in the second component 230 is adapted to receive the LEDs 223, and a second opening 234 in the second component is adapted to receive the light detector 224 and the skin temperature sensor 226. Specifically, when the first component 220 is attached to the second component 230, the emission sides of the LEDs 223 pass through the first opening 232 to allow light emission to be unobstructed. Similarly, when the first component 220 is attached to the second component 230, the detection side of the light detector 224 passes through the second opening 234 to allow light emitted from the LEDs 223 to be incident in an unobstructed manner on the detection side of the light detector 224. The LEDs 223 and the light detector 224 comprise a pulse oximetry sensor that is a component of the integrated sensor 110. As is known, a pulse oximetry sensors is adapted to measure oxygen saturation (SPO2) by passing light through tissue at a location on the body such as a nasal ala to measure light absorption in the blood and thereby measure the oxygen saturation, pulse rate, and site specific perfusion index. Various aspects of the pulse oximetry sensor are described, for example, in the above incorporated U.S. Pat. No. 10,390,715, a copy of which is included in this filing.

With the first component 220 disposed over second component, the ambient sensor is located on an outer surface of the integrated sensor 110 to allow its being located in the ambient region at the application site. The ambient temperature sensor 222 is adapted to measure absolute temperatures of the ambient region near or at the application site.

With the pad 206 disposed over the second component, the skin temperature sensor 226 is adapted to measure absolute temperatures of the skin of the nasal ala against which the pad 206 is disposed.

Because the ambient temperature sensor 222 and the skin temperature sensor 226 according to the present teachings are adapted to measure absolute temperatures, the ambient temperature sensor 222 and the skin temperature sensor 226 may be one or more of a variety of absolute temperature sensors as noted above, and including, but not limited to a thermocouple, a system on a chip, and a silicon/semiconductor temperature sensor.

Finally, it is noted that the ambient temperature sensor 222, LEDs 223 and light detector 224, and the skin temperature sensor 226 are optional components of the integrated sensor 110. While measurements of the SPO2 and temperature from the integrated sensor are advantageous, they are not essentially measured at the nasal ala or in the region thereof, and can be measured elsewhere with other sensors (not shown). Moreover, and as described more fully below, the measurement of the temperature of inhaled air by the temperature sensor 202 provides a fairly accurate estimate of the ambient temperature (e.g., operating room temperature) near the patient. As such, and again while advantageous, the ambient temperature sensor 222 is not essentially a component of the integrated sensor 110.

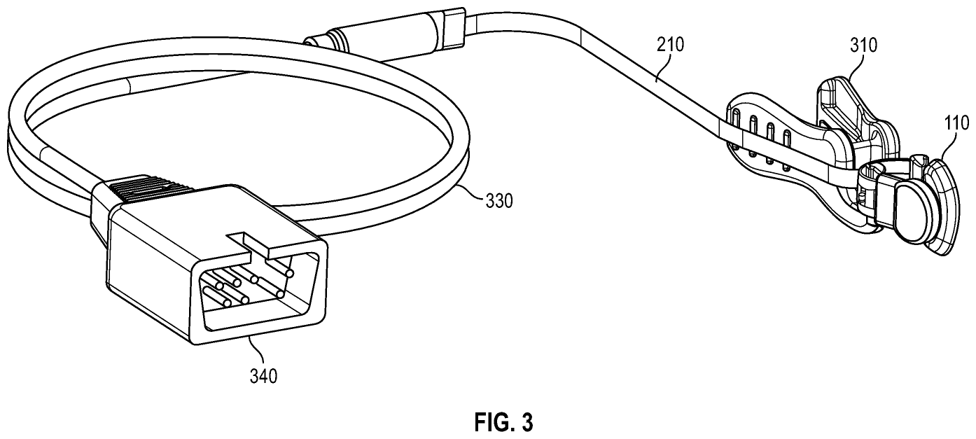

FIG. 3 is a perspective view of an integrated sensor 110 adapted for deployment in accordance with a representative embodiment. Notably, various aspects and details of the integrated sensor 110, and its functionality may be common to those described above in connection with representative embodiments of FIGS. 1-2B. These details may not be repeated below in the interest of clarity of description.

An applicator 310 is attached to the integrated sensor 110 tor foster attachment and detachment of the integrated sensor 110.

The flexible circuit 210 is connected to a cable 330, which is attached to an adapter 340. Notably, the adapter 340 is used to connect the cable 330 to the patient monitor in accordance with a representative embodiment.

The cable 330 is configured to carry the measurements described above from the integrated sensor 110, so that the cable 330 is a single cable and the integrated sensor 110 is a single sensor. As alluded to above, the cable 330 provides electrical signal transmission from the integrated sensor 110 to the patient monitor 140, and electrical power to the integrated sensor 110.

Using the integrated sensor 110 and the cable 330, multiple types of the basic vital signals can be measured at the nasal ala or derived from measurements at the nasal ala, all based on measurements taken by the integrated sensor 110. The basic vital signs include heart rate, respiratory rate, pulse oximetry, temperature, blood pressure and pain. By placing all of the measurements and parameters captured by the integrated sensor 110 over the cable 330, vital signs may be efficiently captured and this may result in a variety of efficiencies such as lower cleaning requirements due to fewer requirements for cables needed to capture vital signs. Efficiencies may also be seen from lower requirements for nurses to spend time inserting, removing, and measuring with temperature probes. Overall costs reductions may also be achieved due to lower requirements for probe covers, probes, thermometers, temperature cables, separate monitors, and adhesives. Moreover, the singularity of the cable 330 helps reduce the number of cables used to monitor and treat patients.

FIG. 4 shows a graph 400 of absolute temperature versus time gathered from measurements from the integrated sensor 110, in accordance with a representative embodiment. Notably, various aspects and details temperature measurements depicted in FIG. 4 may be common to those described above in connection with representative embodiments of FIGS. 1-3. These details may not be repeated below in the interest of clarity of description.

The temperature measurements depicted in FIG. 4 may be gathered only from the temperature sensor 202, and can be used to determine the a respiration rate of a patient; an estimated core (internal) temperature of a patient; and an estimated room temperature at or near the application site of the integrated sensor.

Maximum measured temperatures 402, 404 are the temperatures of air exhaled by a patient from the nasal passage where the integrated sensor 110 is deployed. As will be appreciated, the air that is exhaled has been warmed by the patient's body and is at a maximum level. By contrast, minimum measured temperatures 401, 403 are the temperatures of air inhaled by a patient from the nasal passage where the integrated sensor 110 is deployed. As will be appreciated, the air that is inhaled from the ambient (which is presumed to be less than body temperature) has not been warmed by the patient's body and is at a minimum level.

During deployment, the temperature sensor 202 of the integrated sensor 110 measures the maximum temperature (at 402, 404) at the peak of exhalation of the air from the nasal passage of the patient. Similarly, the temperature sensor 202 of the integrated sensor measures a minimum temperature when air from the ambient is inhaled and is not heated. As will be appreciated, the temperature of the exhaled air from the nasal passage can be used to estimate the internal or core temperature of the patient, as the air exhaled from the lungs has been heated by the body while in the lungs.

A direct correlation between the temperature of the exhaled air and the core temperature can be used to estimate the core temperature based on the maximum measured temperatures. This correlation may be in the form of a lookup table stored in the memory 116 and accessed by the processor 118 to be provided on the display 120 of the patient monitor 112. Alternatively, an algorithm stored can be executed by the processor 118 as instructions to estimate the core temperature based on maximum temperature exhaled by the patient.

Similarly, a direct correlation between the temperature of the inhaled air and the ambient temperature can be used to estimate the ambient temperature at the application site based on the minimum measured temperatures. This correlation may be in the form of a lookup table stored in the memory 116 and accessed by the processor 118 to be provided on the display 120 of the patient monitor 112. Alternatively, an algorithm stored can be executed by the processor 118 as instructions to estimate the ambient temperature based on minimum temperature of air inhaled by the patient.

Finally, the respiration rate, which is the frequency of initial inhalations can be readily determined from the temperatures of inhaled air from a patient. Specifically, at the minimum measured temperature 401, the air has not been warmed by the body and represents an initial inhalation. At minimum measured temperature 403, the second inhalation is recorded, and the respiration rate/frequency of respiration can be readily determined by the processor 118. Alternatively, maximum measured temperatures 402, 404 could be used to measure the frequency of exhalations to determine the respiratory rate by the processor 118.

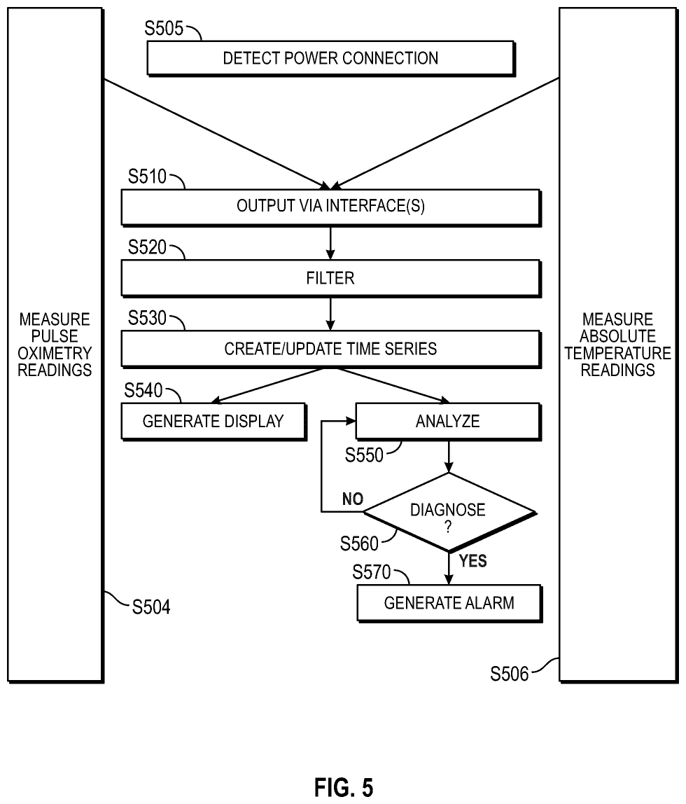

FIG. 5 is a flow-chart of a method for integrated nasal ala SPO2 and temperature sensing, in accordance with a representative embodiment. Notably, various aspects and details of the integrated sensor 110, and its functionality may be common to those described above in connection with representative embodiments of FIGS. 1-2B. These details may not be repeated below in the interest of clarity of description.

The method, and variations thereof, are contemplated to be stored as instructions in memory 116. When executed by the processor 118, the instructions cause the processor 118 to carry out the method. Moreover, not all aspects of the method described below must be carried out. To this end, one method for sensing medical characteristics using the integrated sensor 110 comprises: measuring a temperature of inhaled air entering a nasal passage at the application site; measuring a temperature of exhaled air exiting the nasal passage at the application site; and based on the temperature of the inhaled air, and the exhaled air, determining one of more of: a respiration rate; an estimated core temperature; and an estimated room temperature. As described above, the estimated temperatures can be determined using only the absolute temperature measurements from the temperature sensor 202.

In FIG. 5, the method starts at S505 by detecting a power connection. For example, at S505 a cable connection with a nasal SPO2 sensor of the integrated sensor 110 sensor may be connected to a patient monitor 140 and the integrated sensor 110 may detect that power is being provided. The nasal SPO2 sensor may be placed to contact the nasal ala, and a temperature sensor or multiple temperature-sensing components of the temperature sensor of the integrated sensor may be placed to contact the inside portion of the nasal ala and the base of the nasal passage.

At S504, pulse oximetry readings are measured. The pulse oximetry readings are measured based on a light sensor sensing light absorption and changes in light absorption in oxygenated or deoxygenated blood.

At S506, temperature readings are measured. The temperature readings may include measurements of internal temperature, ambient temperature, and skin temperature.

At S510, the measured pulse oximetry readings and temperature readings are output via one or more interface(s). The output may include analog signals or digital signals

At S520, the measurements of the measured pulse oximetry readings and temperature readings are filtered. The filtering may involve separating low frequency components from high frequency components of the readings. For example, a raw photoplethysmography (PPG) signal used to measure pulse oximetry readings measurements at the surface of the skin may include high frequency and low frequency components. The high frequency PPG components may be separated from the low frequency PPG components. The low frequency PPG components may be filtered or may be separately used such as to derive a respiratory rate.

At S530, time series are created or updated for the measurements of the measured pulse oximetry readings and temperature readings remaining after the filtering at S520.

At S540, a display is generated for the time series. The display may include magnitudes on a Y axis versus time on an X axis, and the changes evident in the time series may be evident in a single time series and in comparisons between multiple time series.

At S550, the measurements are analyzed. The analysis may include comparisons with thresholds, determinations of derivatives such as first derivatives showing changes in magnitude over time and second derivatives showing acceleration or deceleration in the changes in magnitude over time. The analysis may also include identifying correlations between different time series, such as a rise in absolute internal temperature and a rise in heart rate.

As examples of the analysis at S550, the temperatures measured by the temperature sensor 202 and by a pulse oximeter sensor in FIG. 1 can be used to obtain clinical data. The clinical data obtainable via the temperature sensor 202 includes temperature of air going into the body (ambient temperature), temperature of air leaving the body (internal temperature), skin temperature as the nasal ala, and comparative analysis based on the ambient temperature and the internal temperature. The ambient temperature may be used for pediatric patients who are prone to heat loss and patients with diseases that prevent the body from auto-regulating temperature. Central cooling to prevent brain and heart muscle damage may be controlled more closely based on the data of ambient temperature of air entering the body. The internal temperature of air leaving the body may correlate with core body temperature which currently requires an invasive probe that is potentially painful to insert and/or remove, and which also are damaging to the tissues of compromised patients and require relatively complicated monitoring involving dedicated probes, cables, monitor modules, probe covers and other consumables. The internal temperature of air leaving the body may be used by clinicians in many areas of patient care.

As additional examples of the analysis at S550, measurements from the pulse oximeter sensor and from the temperature sensor 202 in FIG. 1 may be used to derive a respiratory rate. For example, a correlation between changes in one or more of the temperatures measured by the temperature sensor 202 and measurements from the pulse oximeter sensor may reflect a respiratory rate and a change in the respiratory rate.

Comparative analysis at S550 may also be performed based on the ambient temperature and the internal temperature and may be used to derive numerous features, including comparisons of time series for the ambient temperature and the internal temperature, and how ambient temperature and internal temperature change over time. For example, a peak temperature may correlate to core temperature. A trough temperature may correlate to ambient room temperature. A median temperature may correlate to a skin temperature at the nasal ala, and this may be used in a predictive model used for acute care in hospitals. An example of correlations between respiratory rate and various temperatures is shown in FIG. 4. The analysis at S550 may be applied to measurements from the integrated sensor 110 or from the integrated sensor 110 combined with other data from other sensors or inputs. As described herein, a number of the vital signs may be measured or derived from the measurements by the integrated sensor 110 or from the integrated sensor 110.

At S560, a determination is made as to whether the measurements result in a diagnosis. If no diagnosis results (S560=No), the method of FIG. 6 returns to S550 for more analysis based on the updated time series from S530. A diagnosis is not necessarily identification of a specific health condition but may be a determination of a health concern that warrants monitoring, or an upgrade in monitoring. For example, a diagnosis may be that oxygenation has decreased below a threshold that warrants increased monitoring, or that absolute internal temperature is rising and heart rate is increasing or decreasing.

If a diagnosis results at S560 (S560=Yes), at S670 the method of FIG. 6 includes generating an alarm. An alarm may simply be a warning on a patient monitor or may include issuing a communication such as a message over an internal computer network or to a mobile device such as a cell phone or tablet issued to a specific medical professional. An alarm may also be a more aggressive alarm, such as if pulse oximetry readings quickly decrease from 98 to 89 such that a medical professional should immediately respond.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although integrated sensor has been described with reference to several exemplary embodiments, it is understood that the words that have been used are words of description and illustration, rather than words of limitation. Changes may be made within the purview of the appended claims, as presently stated and as amended, without departing from the scope and spirit of integrated sensor in its aspects. Although integrated sensor has been described with reference to particular means, materials and embodiments, integrated sensor is not intended to be limited to the particulars disclosed; rather integrated sensor extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A monitoring system, comprising:
an integrated sensor comprising a temperature sensor configured to measure a temperature of inhaled air entering a nasal passage at the application site, and of exhaled air exiting the nasal passage at the application site;
a processor; and
a memory that stores instructions, which, when executed by the processor, cause the monitoring system to determine, based on the temperature of the inhaled air, and the exhaled air one of more of: a respiration rate; an estimated core temperature; and an estimated room temperature.

2. The monitoring system of claim 1, wherein the integrated sensor further comprises an ambient air temperature sensor adapted to measure an ambient air temperature at an application site.

3. The monitoring system of claim 1, wherein the integrated sensor further comprises a pulse oximeter configured to measure oxygen saturation at the application site.

4. The monitoring system of claim 1, wherein the integrated sensor further comprises a skin temperature sensor adapted to measure a temperature of skin.

5. The monitoring system of claim 1, further comprising a connection configured to transmit signals from the temperature sensor.

6. The monitoring system of claim 5, wherein the connection comprises one or more of:
an electrical signal transmission line;
an optical fiber and an electrical power line; and
a wireless link.

7. The monitoring system of claim 1, wherein the temperature sensor does not measure a relative temperature.

8. The monitoring system of claim 1, wherein the memory and the processor are disposed in an intermediary device between the temperature sensor and a patient monitor.

9. The monitoring system of claim 1, wherein the memory and the processor are disposed in a smart cable device connected to the temperature sensor.

10. The monitoring system of claim 1, wherein the memory and the processor are disposed in a patient monitor.

11. The monitoring system of claim 3, wherein the pulse oximeter sensor and the temperature sensor are physically integrated into a single unit.

12. An integrated sensor, comprising:
an integrated sensor comprising a temperature sensor configured to measure a temperature of inhaled air entering a nasal passage at the application site, and of exhaled air exiting the nasal passage at the application site; and
an adapter that outputs measurements of the temperature of the exhaled air, and the temperature of the inhaled air.

13. The integrated sensor of claim 12, wherein the integrated sensor further comprises an ambient air temperature sensor adapted to measure an ambient air temperature at an application site.

14. The monitoring system of claim 12, wherein the integrated sensor further comprises a pulse oximeter configured to measure oxygen saturation at the application site.

15. The monitoring system of claim 12, wherein the integrated sensor further comprises a skin temperature sensor configured to measure a temperature of skin at the nasal ala.

16. A method for sensing medical characteristics using an integrated sensor-, the method comprising:
measuring a temperature of inhaled air entering a nasal passage at the application site;
measuring a temperature of exhaled air exiting the nasal passage at the application site; and
based on the temperature of the inhaled air, and the exhaled air, determining one of more of: a respiration rate; an estimated core temperature; and an estimated room temperature.

17. The method of claim 16, further comprising:
measuring an ambient temperature at the application site; and
determining the estimated room temperature based on the ambient temperature.

18. The method of claim 16, wherein the measuring of the measuring a temperature of inhaled air entering the nasal passage at the application site, or the measuring the temperature of exhaled air exiting the nasal passage at the application site, or both, are done continuously or continually over time.

19. The method of claim 16, further comprising measuring oxygen saturation at the application site.

20. The method of claim 16, wherein the measuring of the measuring a temperature of inhaled air entering a nasal passage at the application site and the measuring a temperature of exhaled air exiting the nasal passage at the application site are done continuously or continually over time.

21. A tangible, non-transitory computer readable storage medium that stores instructions, which, when executed by a processor, cause the processor to:
receive temperature measurements of inhaled air from a nasal passage at an application site, and of exhaled air from the nasal passage at the application site; and
determine, based on the temperature of the inhaled air, and of the exhaled air, one of more of: a respiration rate; an estimated core temperature; and an estimated room temperature.

22. The tangible, non-transitory computer readable storage medium of claim 21, wherein the instructions, which, when executed by the processor, further cause the processor to:
receive an ambient temperature at the application site; and
determine the estimated room temperature based on the ambient temperature.

23. The tangible, non-transitory computer readable storage medium of claim 21, wherein the temperature measurements of the inhaled air and of the exhaled air are taken continuously or continually.

24. The tangible, non-transitory computer readable storage medium of claim 21, wherein the instructions, when executed by the processor, further cause the processor to:
receive oxygen saturation measurements at an application site; and
determine an oxygen saturation measurement time series of the oxygen saturation measurements.
